(19) United States
(12) Patent Application Publication
(10) Pub. No.: US 2004/0122397 A1
(43) Pub. Date: Jun. 24, 2004

(54) Absorbent garment having a trapezoidal shaped retention element

(76) Inventors: Michael T. Morman, Alpharetta, GA (US); Paul T. Van Gompel, Hortonville, WI (US); Jennifer L. Marvin, Greenville, WI (US); Thomas H. Roessler, Appleton, WI (US); James M. Carr, Kaukauna, WI (US); Yung H. Huang, Appleton, WI (US); Mary Jo Meyer, Neenah, WI (US); Eric D. Johnson, Larsen, WI (US)

Correspondence Address: GENERAL NUMBER 00757, BRINKS HOFER GILSON & LIONE, P.O. BOX 10395, CHICAGO, IL, 60611, US

(21) Appl. No.: 10/325,625

(22) Filed: Dec. 19, 2002

(51) Int. Cl. A61F 13/15; A61F 13/20

(52) U.S. Cl. 604/385.01

(57) ABSTRACT

An absorbent garment includes a front body panel having a terminal waist edge and a terminal crotch edge and a rear body panel having a terminal waist edge and a terminal crotch edge. A crotch member extends between and is coupled to the front and rear body panels. The crotch member includes a retention element having a trapezoidal shape. In another aspect, a method of manufacturing an absorbent garment includes moving first and second webs of body panel material in a first machine direction, moving a web of absorbent material in a second machine direction, and cutting the web of absorbent material and thereby forming a plurality of trapezoidally shaped retention elements, with each of the plurality of retention elements having non-parallel side edges. The method further includes successively coupling the plurality of retention elements to the first and second webs with the side edges extending in a cross-direction.

### BACKGROUND

The present invention relates generally to absorbent garments, and in particular, to an absorbent garment having a trapezoidal shaped retention element, and to methods for the manufacture thereof.

Absorbent garments can be configured in many different forms. For example, absorbent garments can be configured with a body chassis that extends from the front to the back of the user. Typically, such garments have a retention element, made of an absorbent material, which is supported by a crotch region of the body chassis. Often these types of garments are manufactured in the machine direction, with the retention element extending along the machine direction. Other absorbent garments have separate front and rear body panels that are spaced apart in the crotch region. A crotch member, which typically includes a retention element, is secured to the front and rear body panels and bridges the space therebetween.

Typically, the retention element used in such garments is configured in a rectangular shape, or alternatively, in an hour-glass shape. For example, when manufactured in the machine direction, the retention element can simply be cut from a roll to define a machine direction length thereof. The retention element is thereafter placed on the garment. To avoid waste, the retention portion is typically rectangular, i.e., configured with straight sides. However, the rectangular retention portion may be bulky in the crotch region, and in particular at the location between the legs of the user, while not providing maximum protection in the rear of the garment.

Alternatively, the retention portion can be cut, for example in the hour-glass shape, to improve the fit of the garment to the user as it narrows in the crotch region, while providing greater surface area coverage in the front and rear of the garment. Typically, however, some retention material waste is generated due to the cut-out shape of the retention element.

Therefore the need remains for improved absorbent garments that provide maximum conformance and absorbent coverage with improved fit while reducing or eliminating waste material during the manufacturing process.

### SUMMARY

Briefly stated, in one embodiment, an absorbent garment includes a front body panel having a terminal waist edge and a terminal crotch edge and a rear body panel having a terminal waist edge and a terminal crotch edge. A crotch member extends between and is coupled to the front and rear body panels. The crotch member includes a retention element having a trapezoidal shape.

In another aspect, one embodiment of a method of manufacturing an absorbent garment includes moving first and second webs of body panel material in a first machine direction, moving a web of absorbent material in a second machine direction, and cutting the web of absorbent material and thereby forming a plurality of trapezoidally shaped retention elements, with each of the plurality of retention elements having non-parallel side edges. The method further includes successively coupling the plurality of retention elements to the first and second webs with the side edges extending in a cross-direction relative to the first machine direction.

In one embodiment, the method further includes successively rotating every other one of the plurality of retention elements approximately 180 degrees about an axis other than a cross direction axis and successively coupling the retention portions to the first and second webs with the side edges extending in a cross-direction, wherein the retention portion bridges at least a portion of the gap between the first and second webs.

The various embodiments provide significant advantages over other absorbent garments and methods of manufacture. For example, the trapezoidal shaped retention element has a lesser width in the crotch region than in the buttocks region, so as to provide an improved fit with increased buttocks coverage. At the same time, the retention elements can be cut from a single web of material so as to eliminate any waste material.

The foregoing paragraphs have been provided by way of general introduction, and are not intended to limit the scope of the following claims. The presently preferred embodiments, together with further advantages, will be best understood by reference to the following detailed description taken in conjunction with the accompanying drawings.

### BRIEF DESCRIPTION OF THE DRAWINGS

FIG. 1 is a plan view of a first embodiment of an absorbent garment.

FIG. 2 is a cross-sectional cut of the crotch member taken along line 2-2 of FIG. 1.

FIG. 3 is a plan view of a second embodiment of an absorbent garment.

FIG. 4 is a cross-sectional cut of the crotch member taken along line 2-2 of FIG. 1.

FIG. 5 is a schematic illustration of one method of manufacturing absorbent garments.

FIG. 6 is a schematic illustration of an alternative method of manufacturing absorbent garments.

### DETAILED DESCRIPTION OF THE PRESENTLY

### PREFERRED EMBODIMENTS

It should be understood that the term “longitudinal,” as used herein, means of or relating to length or the lengthwise direction, for example the lengthwise direction of the absorbent garment. The term “laterally,” as used herein, means situated on, directed toward or running from side to side, for example from side to side of the absorbent garment.

The term “bodyside” should not be interpreted to mean in contact with the body of the user, but rather simply means the side that would face toward the body of the user, regardless of whether the absorbent garment is actually being worn by the user and regardless of whether there are or may be intervening layers between the component and the body of the user. Likewise, the term “garment side” should not be interpreted to mean in contact with the garments of the user, but rather simply means the side that faces away from the body of the user, and therefore toward any outer garments that may be worn by the user, regardless of whether the absorbent garment is actually being worn by a user, regardless of whether any such outer garments are actually worn and regardless of whether there may be intervening layers between the component and any outer garment.

The term “machine direction” means the direction of flow as the various members and webs progress along a fabrication line and process. It should be understood that various separate members or webs can each be traveling in a machine direction, but with the various machine directions not necessarily being parallel or oriented in the same direction. For example, a first component such as a web may be traveling in a first machine direction, which is substantially perpendicular to the travel of another component, such as a retention element, in a second machine direction.

The term “cross direction” means the direction substantially perpendicular to the machine direction.

The term “downstream” means that one item is positioned more closely to the output or finished product end of the machine and/or process relative to another item. Conversely, the term “upstream” means that an item is positioned more closely to the input end of the machine or process relative to another item. For example, the output end is downstream of the input end, and vice versa, the input end is upstream of the output end.

The phrases “removeably attached,” “removeably attaching,” “removeably connected,” “removeably engaged,” “releasably attached,” “releasably connected,” or “releasably engaged,” and variations thereof, refers to two or more elements being connected or connectable such that the elements tend to remain connected absent a separation force applied to one, both or all of the elements, and where the elements are capable of being separated upon the application of a separation force. The required separation force is typically beyond that encountered while wearing the absorbent garment.

The phrases “fixedly secured,” “fixedly engaged,” “fixedly attached,” “fixedly connected,” and variations thereof, refers to two or more elements being connected or connectable such that they are not disconnected or otherwise separated, and are not intended to be separated or disconnected, during the normal operation and use of the absorbent garment.

The term “web” refers to a continuous stream of material, whether made from one or more layers or substrates, and regardless of whether it may have non-continuous, discrete items disposed thereon.

The terms “connecting,” “coupled,” “attached,” and “secured,” and variations thereof, broadly covers two or more items being directly connected one to the other, or by way of one or more intervening members or components.

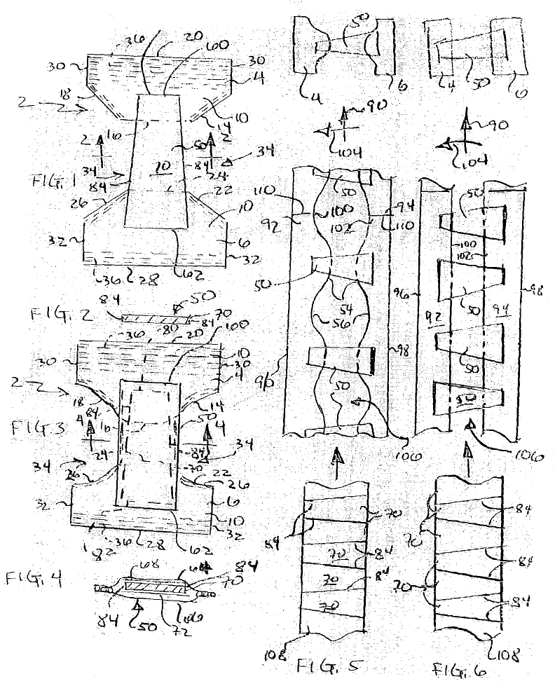

Referring to FIGS. 1 and 3, an absorbent garment 2 includes a first, front body panel 4 and a second, rear body panel 6. The term “body panel” refers to the portion(s) of the absorbent garment, whether made of one or more layers or substrates or of one or more pieces or components, that is/are fitted circumferentially around at least the waist region of the user, including for example the user's lower back, buttocks, hips and abdomen. The first and second body panels each have an inner, bodyside surface 10 and an outer, garment side surface. The first, front body panel 4 has a first edge 14 forming a crotch portion 16 and leg opening portion 18 and a second terminal edge 20 that is preferably linear but can assume other shapes. Likewise, the second, rear body panel 6 has a first edge 22 forming a crotch portion 24 and a leg opening portion 26 and a second terminal edge 28, which is linear in one embodiment but can assume other shapes. Each of the first and second body panels has an outboard side edge 30, 32 formed along the outer periphery of the opposite side portions of the first and second body panel. It should be understood that the outboard side edges of the front and rear body panels can have different lengths relative to each other.

Referring to FIGS. 1 and 3, one or more, and in one embodiment a plurality, meaning two or more, elastic elements 36 are secured to each of the first and second body panels. In one exemplary embodiment, a plurality of elastic elements are spaced across substantially the entirety of the front and rear body panel 4, 6, although they may be spaced across a lesser length, or along various predetermined, selected regions of the body panels. For example, elastic elements can extend along the upper waist portion and along the lower terminal edge defining in part a leg opening 34.

In one embodiment, the front body panel has a “non-elasticized” area wherein there are no elastic elements, or other elastic or elastomeric backing members, incorporated therein or making up any portion of the thickness or cross-section of the body panel at that area. It should be understood, that in an alternative embodiment, one or more separate waist bands, with or without elastic elements, can be secured to one or both of the rear and front body panels, preferably along the upper terminal edges 20, 28 thereof. Likewise, one or more separate leg bands can be secured to one or both of the rear and front body panels along the leg open portions 18, 26 adjacent the leg openings 34. Alternatively, one or both of the body panels can be formed without any elastic elements.

The various waist and leg elastic elements can be formed from rubber or other elastomeric materials. One suitable material is a LYCRA® elastic material. For example, the various elastic elements can be formed of LYCRA® XA Spandex 540, 740 or 940 decitex T-127 or T-128 elastics available from E.I. duPont De Nemours and Company, having an office in Wilmington, Del.

In one embodiment, each body panel 4, 6 is formed as a composite, or laminate material, otherwise referred to as substrates or laminates, with the plurality of elastic strands sandwiched therebetween. For example, two or more layers may be bonded with various adhesives, such as hot melt, or by other techniques, including for example and without limitation ultrasonic bonding and heat pressure sealing. In one embodiment, the two layers are made of a non-woven material such as a spunbond material, a bonded carded material or other known materials. It should be understood that the body panels can be made of a single layer or substrate of non-woven material, or can be comprised of more than two layers or substrates. Of course, it should be understood that other knitted or woven fabrics, non-woven fabrics, elastomeric materials, polymer films, laminates and the like can be used to form one or more of the body panel layers. The term “non-woven” web or material, as used herein, means a web having a structure of individual fibers or filaments that are interlaid, but not in an identifiable manner and without the aid of textile weaving or knitting, as in a knitted or woven fabric.

In one embodiment, the body panel material can be secured to the elastic elements, such as strands or ribbons, which have been elongated and retracted, such that the material is gathered when the elastic elements are relaxed. Alternatively, the material can be gathered and laminated to non-elongated elastic elements. In one embodiment, the body panel includes a gathered elastic laminate made from nonwoven base sheets bonded with elongated elastic elements sandwiched therebetween.

In various embodiments, the body panel material may be substantially permeable to air or substantially impermeable to air. The body panel material also may be substantially liquid-permeable or substantially liquid-impermeable. In particular arrangements, the body panel material may be substantially nonelastomeric. In other aspects, the body panels can include an elastomeric material that is elastomerically stretchable at least along the lateral article width. Examples of such elastomeric composite materials can include a vertical filament laminate (VFL), neck-bonded-laminate (NBL), a stretch-bonded-laminate (SBL), a necked-stretch bonded laminate (NSBL) or a necked-thermal laminate, or the like, as well as combinations thereof. Exemplary NBL, SBL, and NSBL materials are described in U.S. Pat. Nos. 5,226,992, 4,981,747, 4,965,122, 5,336,545, 5,385,775, 5,414,470, 4,720,415, 4,789,699, 4,781,966, 4,657,802, 4,652,487, 4,655,760, 5,116,662 and 5,114,781, all of which are hereby incorporated herein by reference. Exemplary VFL materials are described in U.S. Provisional Patent Application Serial No. 60/204,307, filed May 15, 2000 and entitled “Method and Apparatus for Producing Laminated Articles,” and PCT application WO 01/88245 A2, both assigned to Kimberly-Clark Worldwide, Inc., the assignee of the present application, with the entire disclosures of both being hereby incorporated herein by reference. Such laminates can provide an improved combination of cloth-like feel and elastomeric stretchability. The body panels can be composed of materials that are elastic or elastomeric and exhibit biaxial stretch characteristics or MD/CD stretch characteristics, or that are extensible composites. Additional waist and leg elastic elements can be added to, but are not necessarily required by, the body panels.

In one embodiment, the entirety of the body panels are elasticized, such that the entire body panel conforms to the body of the user with minimal (including zero) spacing between the body panel and the user's body, and without the attendant bulkiness of a non-elasticized material.

In one embodiment, the body panels are breathable, cloth-like, nonwoven laminates with multi-directional stretch and/or extensible properties. In one embodiment, the non-woven layers are pre-necked in the cross direction, for example between about 90% and about 20% of the original width, which provides extensibility in the longitudinal direction of the absorbent garment with minimum force.

The terms “extensible,” “extensibility,” and variations thereof as used herein means capable of being extended, and providing a selected elongation, for example between about 5% and about 70%, when subjected to an applied tensile force. The body panel also is preferably capable of providing a selected, sustained deformation when subjected to an applied tensile force and then allowed to relax for a selected time period beginning immediately after removal of the tensile force. Preferably the sustained deformation is a substantially permanent deformation. The selected elongation and sustained deformation preferably occur at least along the longitudinal direction of the garment, although it should be understood that it also could occur along the lateral direction, or both. Various extensible materials, and other acceptable materials that can be used for the body panels are described for example in U.S. Pat. No. 6,217,563, issued Apr. 17, 2001 to Kimberly-Clark Worldwide, Inc., the same Assignee as the present application, the entire disclosure of which is hereby incorporated herein by reference.

The extensibility of the non-woven material provides an increase in surface area without the retractive force of elastomeric materials. In one embodiment, body panel is extensible in at least the cross direction, or the longitudinal direction of the absorbent garment, with the material providing an elongation of at least about 1 cm when subjected to a tensile force of 11.8 grams per cm. In addition, the body panel preferably provides a substantially permanent deformation of at least about 20% when it is subjected to a tensile force of 19.70 grams per cm and is then allowed to relax under a zero applied force for a period of 1 minute. Of course, it should be understood that the body panel can also be made extensible in the lateral direction.

In one embodiment, the front and rear body panels 4, 6 are made of non-woven laminates of two layers of longitudinally extensible 0.60 osy polypropylene spunbond material with elongated strands of Lycra® elastic sandwiched between the spunbond layers and thereafter adhesively bonded. In particular, the body panel material is necked in the cross direction. As used herein, the term “necked,” and variations thereof, refers to any material that has been constricted in at least one dimension by applying a tensioning force in a direction that is perpendicular to the desired direction of neck-down. Processes that may be used to constrict a material in such a manner include, for example and without limitation, drawing processes. For example, cross direction necking provides the body panel material with longitudinal direction extension. In one embodiment, the elastics are elongated in the machine direction and secured to the body panel material. The elastics are then allowed to retract so as to gather the necked spunbond material in the machine direction thereby creating an elastically gathered non-woven body panel with lateral extensibility. The term “gather,” and variations thereof, as used herein means puckered, or contracted into folds or wrinkles, which should be understood as including micro-pleats. In this way, the body panel can be elongated in both the longitudinal and lateral direction to conform to the body of the user when the garment is applied thereto. In particular, as the user pulls the garment up over their hips, the non-woven laminate body panels stretch in the lateral direction while the leg regions of the front and rear body panels conform to the crotch and body lines of the user. At the same time, the body panel material extends in the longitudinal direction to conform to the buttocks and stomach of the user. The extensibility of the body panels follows the natural curvature of user's body to provide conformance thereto. As the body panel extends in the longitudinal direction, the spacing between the laterally extending elastic elements 36, incorporated in one preferred embodiment, will increase.

In one embodiment, the body panel 4, 6 non-woven material is substantially hydrophobic, which may optionally be treated with a surfactant or otherwise processed to impart a desired level of wettability and hydrophilicity. In one particular embodiment of the invention, the body panel is a nonwoven, wireweave spunbond polypropylene fabric composed of about 1.6 denier fibers formed into a web having a basis weight of about 0.6 osy. One suitable non-woven material is the Corinth 0.60 osy, 1.6 dpf wireweave, nonwettable Metallocene (EXXON ACHIEVE 2854 PP) spunbond material manufactured by Kimberly-Clark Corporation, the assignee of the present application.

It should be understood that in various embodiments, one or both of the front and rear body panels can be non-extensible and non-elastic, extensible and non-elastic, or extensible and elastic, and various combinations thereof. For example, one or both of the front and rear body panels can be formed in part from a non-elastic, non-extensible film.

Referring to FIGS. 1 and 3, a crotch member 50 connecting the front and rear body panels 4, 6 can be folded such that the side edges 30, 32 of the front and rear body panels 4, 6 are aligned wherein they can be fixedly secured at a seam. The seam can be formed by bonding, sewing or otherwise attaching the side edges. Alternatively, the product can remain “open,” wherein the body panels are releasably secured with one or more fastening members as explained below.

In one embodiment the garment includes a combination of side edges that are secured to form a seam and fastening members (not shown) that allow the fit of the absorbent garment to be adjusted. For example, fastening members can be attached to the front body panel and extend inboard relative to the outboard side edge 30 of the front body panel 4 from an attachment location, which is spaced inboard from the side edge. A landing member (not shown) can be formed on or secured to the body panel to receive a refastenable portion of the fastening member. One or more lines of weakness can be provided along the front or rear body panel such that one or both of the body panels are breakable. The lines of weakness can comprise a perforation or other series of cuts, a thinning, breakage or separation of material, or a strip of a different kind of material bridging portions of the body panel that is more easily torn or broken than the other material thereof, which allow a user or the manufacturer to separate portions of the body panel. For example, the absorbent garment can be broken along the lines of weakness after the garment is applied to a user, or beforehand. In one embodiment, the fastening members are secured to the garment-side surface of the body panel.

It should be understood that, in other embodiments, the fastening members can be secured to the rear body panel and engage the front body panel or, conversely, can be secured to the front body panel and engage the rear body panel, for example along at least a portion that is not elasticized. In one embodiment, the fastening members are fixedly secured to the outer, garment-side surface of the front and/or rear body panels, and releasably engage the outer, garment-side surface or the inner, body-side surface, of the front and/or rear body panels. In other embodiments, the fastening members are fixedly secured to an inner body-side surface of front and/or rear body panels and releasably engage an inner, body-side surface, or an outer, garment side surface, of the front and/or rear body panels.

When incorporated into an absorbent garment, the fastening members preferably include a refastenable portion, such as an array of hook members, adhesives, such as pressure sensitive adhesives, buttons, zippers, snaps and other releasable and reattachable fastening devices. In various preferred embodiments, the fastening member includes one, two or more than two tab members. In one embodiment, the fastening members comprise a carrier member, which is fixedly secured to the side portions of the front body panel with adhesive bonds, sonic bonds, thermal bonds, pinning, stitching or other known types of attachment. In alternative embodiments, the fastening members can be fixedly secured to the rear body panel or to one or both of the front and rear body panels, for example, at the seam, as explained above.

Referring to FIGS. 1 and 3, the absorbent garment includes a crotch member 50 having first and second opposed terminal end edges 60, 62. In one embodiment, shown in FIGS. 3 and 4, the crotch member includes a substantially liquid permeable topsheet 64, or liner, and a substantially liquid impermeable backsheet 66, or outer cover. A retention element 70 is disposed or sandwiched between the topsheet and the backsheet, which are connected. The topsheet, backsheet and other components of the crotch member 50 can be joined for example with adhesive bonds, sonic bonds, thermal bonds, pinning, stitching or any other attachment techniques known in the art, as well as combinations thereof. For example, a uniform continuous layer of adhesive, a patterned layer of adhesive, a sprayed pattern of adhesive or any array of lines, swirls or spots of construction bonds may be used to join the topsheet and backsheet, or any of the other components described herein. It should be understood that the term “retention element” refers to any material or assembly capable of absorbing liquids or bodily exudates, and may be comprised of a single material or component, or can be formed as a composite of several components.

Additional layers, including for example, an intake/distribution layer 68 (otherwise referred to as a surge layer or transfer layer), can also be incorporated into the crotch member. In one embodiment, the surge layer does not run the entire length of the crotch member and is shorter than the retention element. The topsheet can be indirectly joined to the backsheet by affixing the topsheet to intermediate layers, such as the surge layer or retention element, which in turn is affixed to the backsheet. The crotch member also may include barrier cuffs, or leakage control shields, formed along the opposite longitudinally extending edges thereof. In addition, as shown for example in FIGS. 3 and 4, elastic elements 38 can be secured to the crotch member, for example between the backsheet and top sheet, along the side edges thereof to provide a gasket with the user's legs.

In one embodiment, the backsheet 66 is liquid impermeable, but may be liquid permeable, e.g., when an additional barrier layer 72 is used with the retention element. For example, in one embodiment, the backsheet can be made from a thin plastic film, or other flexible, substantially liquid-impermeable material. As used herein, the term “flexible” means a material that is compliant and which will readily conform to the general shape and contour of the body of the user. The backsheet prevents various bodily fluids and exudates from wetting or otherwise contaminating various bedding or outer garments worn by the user over the absorbent garment. In particular, the backsheet can include a film, such as a polyethylene film, having a thickness of from about 0.012 mm to about 0.051 mm.

In various constructions, the topsheet 64 can comprise various woven or nonwoven materials. For example, the topsheet can be composed of a meltblown or spunbonded web of desired fibers, and may also be a bonded-carded web. For example, the topsheet can be made of a substantially hydrophobic material, and the hydrophobic material may optionally be treated with a surfactant or otherwise processed to impart a desired level of wettability and hydrophilicity. In one particular embodiment of the invention, the topsheet is a nonwoven, spunbond polypropylene fabric composed of about 1.5-3.2 denier fibers formed into a web having a basis weight of about 22 gsm and density of about 0.06 gm/cc. The fabric can be surface treated with an operative amount of surfactant, such as about 0.28% Triton X-102 or Achovel surfactant. The surfactant can be applied by any conventional means, such as spraying, printing, brush coating or the like.

In various constructions, the backsheet can comprise a woven or nonwoven fibrous web layer, which is treated or constructed, partially or wholly, to impart the desired levels of liquid impermeability to selected regions that are adjacent to or proximate the absorbent retention portion. For example, the backsheet may include a gas-permeable, nonwoven fabric layer laminated to a polymer film layer which may or may not be gas-permeable. Other examples of fibrous, cloth-like backsheet materials can comprise a stretch thinned or stretch thermal laminate material composed of a 0.6 mil (0.015 mm) thick polypropylene cast film and a 0.7 ounce per square yard (23.8 gsm) polypropylene spunbond material (2 denier fibers). A material of this type has been employed to form the outercover of a HUGGIES® Ultratrim Disposable Diaper, which has been commercially available from Kimberly-Clark Corporation. The backsheet can provide the outercover of the article, particularly in the crotch region. Optionally, however, the article may include a separate outercover component member, as disclosed herein, which is additional to the backsheet. The outercover can be joined, for example, to one or more of the absorbent composite and/or body panels as explained above.

The backsheet may include a micro-porous, “breathable” material which permits gases, such as water vapor, to escape from the absorbent garment while substantially preventing liquid exudates from passing through the backsheet. For example, the breathable backsheet may be composed of a microporous polymer film or a nonwoven fabric which has been coated or otherwise modified to impart a desired level of liquid impermeability. For example, a suitable microporous film can be a PMP-1 material, which is available from Mitsui Toatsu Chemicals, Inc., a company having offices in Tokyo, Japan; or an XKO-8044 polyolefin film available from 3M Company of Minneapolis, Minn. The backsheet may also be embossed or otherwise provided with a pattern or matte finish to exhibit a more aesthetically pleasing appearance.

In various configurations of the invention, where a component, such as the backsheet is configured to be permeable to gas while having a resistance and limited permeability to aqueous liquid, the liquid resistant component can have a construction which is capable of supporting a selected hydrohead of water substantially without leakage therethrough. A suitable technique for determining the resistance of a material to liquid penetration is Federal Test Method Standard FTMS 191 Method 5514, 1978, or an equivalent thereof. In one preferred embodiment, the backsheet is sufficiently impermeable to liquid and semi-liquid materials to substantially prevent the undesired leakage of waste materials, defined as exudates, including for example urine and feces. For example, the backsheet member can desirably support a hydrohead of at least about 45 centimeters (cm) substantially without leakage. The backsheet member can alternatively support a hydrohead of at least about 55 cm, and optionally, can support a hydrohead of at least about 60 cm, or more, to provide improved benefits.

The backsheet and/or outercover also can be extensible and/or elastic, in the lateral or longitudinal directions. In one embodiment, the backsheet and/or outercover is capable of providing an elongation of at least about 1 cm when subjected to a tensile force of 11.8 g/cm, and further provides a substantially permanent deformation of at least about 20% when subjected to a tensile force of 19.70 g/cm and is then allowed to relax under a zero applied force for a period of 1 minute.

For example, the extensible member can be composed of necked fibers, creped fibers, micro-pleated fibers, polymer films or the like, as well as combinations thereof. The fabrics may be woven or nonwoven materials, such as spunbond fabrics. One example of a suitable extensible material is a 60% necked, polypropylene spunbond having a necked basis weight of about 1.2 osy.

The backsheet and/or outercover also can be expandable, for example when it has one or more folds, e.g., one or more z-folds (not shown), or can be both extensible and expandable. The term expandable as used herein means to enlarge or to increase the extent or area, lateral and/or longitudinal, thereof, e.g., by unfolding one or more folds.

The retention element 70 is made of an absorbent material. For example, the absorbent material can be made of airformed, airlaid and/or wetlaid composites of fibers and high absorbency materials, referred to as superabsorbents. The retention element has an absorbent capacity of greater than about 7 grams/gram at a 0.50 psi loading. The overall absorbent capacity is greater than about 50 grams, and in various embodiments greater than 100 grams.

Superabsorbents typically are made of polyacrylic acids, such as FAVOR 880 available from Stockhausen, Inc. of Greensboro, N.C. The fibers can be fluff pulp materials, such as Alliance CR-1654, or any combination of crosslinked pulps, hardwood, softwood, and synthetic fibers. Airlaid and wetlaid structures typically include binding agents, which are used to stabilize the structure. In addition, various foams, absorbent films, and superabsorbent fabrics can be used as an absorbent material. Various acceptable absorbent materials are disclosed in U.S. Pat. No. 5,147,343 for Absorbent Products Containing Hydrogels With Ability To Swell Against Pressure, U.S. Pat. No. 5,601,542 for Absorbent Composite, and U.S. Pat. No. 5,651,862 for Wet Formed Absorbent Composite, all of which are hereby incorporated herein by reference. Furthermore, the proportion of high-absorbency particles can range from about 0 to about 100%, and the proportion of fibrous material from about 0 to about 100%. Additionally, high absorbency fibers can be used such as Oasis type 121 and type 122 superabsorbent fibers available from Technical Absorbent Ltd., Grimsby, Lincolnshire, United Kingdom.

In various embodiments, the retention element 70 can be made of a single or dual layer of absorbent material. Alternatively, the retention element 70 can include a folded or multi-layered configuration. The retention element preferably has a length substantially equal to, or slightly shorter than, the length of the absorbent insert. The retention element 70 can include one or-more barrier layers 72 attached to the absorbent material. In one embodiment, an upper tissue substrate is disposed adjacent the retention element. Alternatively, a lower tissue substrate can be disposed adjacent an opposite side of the retention element, or the tissue can completely envelope the retention element.

As shown in FIGS. 1 and 3, the retention element 70 has a trapezoidal shape with first and second parallel terminal edges 80, 82 and non-parallel, linear side edges 84, wherein said first terminal edge is shorter than said second terminal edge. As shown in FIGS. 1 and 3, the first terminal edge 80 overlies the front body panel 4 and the second terminal edge 82 overlies the rear body panel 6. Of course, it should be understood that the opposite orientation could also be used. In the embodiment of FIG. 3, the retention element 70 preferably has a length substantially equal to, or slightly shorter than, the length of the crotch member 50. In one embodiment, the first terminal edge is between about 0.50 inches and about 3.00 inches, while the second terminal edge is between about 1.00 inches and about 5.00 inches. The overall length, in one embodiment, is between about 3 inches and about 15 inches. Of course, it should be understood that the retention element can have other dimensions not specifically enumerated herein.

Referring to FIG. 3, the opposite garment side of the end regions of the absorbent crotch member 50, and in particular, the outer, garment side surface of the backsheet, are secured to the bodyside surface of the opposed crotch portions of the first and second body panels 4, 6. Alternatively, the bodyside surface of the crotch member is secured to the garment side surface of the body panels. In the embodiment shown in FIG. 3, the backsheet and top sheet can have a rectangular shape, or they can have a trapezoidal shape that is oriented to correspond to the trapezoidal shape of the retention element.

Referring to FIG. 1, the crotch member 50 includes a retention element 70 without a topsheet or backsheet, and can be formed entirely of the retention portion. In such embodiments, the retention element is secured directly to the first and second body panels.

In either embodiment of FIG. 1 or FIG. 3, it should be understood that the crotch member 50 can be secured using any of the methods of attachment described above, including for example various adhesives, stitching or other bonding methods. The crotch member can be secured to the body panels with any configuration of attachment lines, swirls, patterns, spots, etc., or can be a full and continuous attachment therebetween.

The entire portion of the crotch member 50 overlapping the body panels 4, 6 can be attached thereto, or the crotch member can be minimally attached to the body panels, for example by one or more lines of attachment formed along the centerline of the absorbent composite, or along a line adjacent the crotch portions of the body panels, so as to allow the body panels to stretch from side to side and extend from front to back, or from the crotch to the waist.

Referring to FIGS. 5 and 6, the method for fabricating one or more embodiments of the aforedescribed absorbent garment is illustrated. Referring to FIG. 5, a web of body panel material is cut in a longitudinal machine direction 90 to form a front and rear body panel web 92, 94. In one embodiment, the web is cut in a sinusoidal wave pattern, which should be broadly interpreted as a pattern having peaks 54 and valleys 56, with the pattern generally defined by a pitch and an amplitude. The pattern can be formed of undulating curves, or can include or be made entirely of various linear portions. Alternatively, as shown in FIG. 6, the web can simply be cut to form linear edges in the machine direction.

Each body panel web includes an outer lateral edge 96, 98 and an inner cut edge 100, 102. In one embodiment, the inner cut edges 100, 102 of the front and rear body panel webs correspond, or mate such that they have the same shape and amplitude. In such an embodiment, no waste material is generated.

In another embodiment, a die cutter (not shown) is used to cut the web. The die cutter is configured to form inner cut edges on the front and rear body panels having different shapes and amplitudes. In such an embodiment, a minimal amount of waste material is generated, but it allows for the front and rear body panels to be specifically shaped to conform to the body of the user. For example, the shape of the rear body panel web cut edge can be provided with lesser amplitude than the amplitude of the front body panel web. In this embodiment, a minimal amount of waste material is produced. In one embodiment, one of the body panels is formed with a sinusoidal cut edge, while the other is configured with a straight cut edge. In various embodiments, the cut edges can be formed by an oscillating cutter, slitters, water jets, lasers and other known cutting devices.

In one embodiment, shown in FIG. 5, each of the front and rear body panel webs 92, 94 has a maximum rise, measured at the peak 54, and a minimum rise, measured at the valley 56. The term “rise,” as used herein, means the measured distance between two edges, for example the outer edge 96, 98 and the inner cut edge 100, 102 of each of the front and rear body panel webs 92, 94 respectively. As shown in FIGS. 1 and 3, the total rise of the garment is measured between the outer edges 20, 28 of the front and rear body panels, or body panel webs, after the absorbent insert is connected thereto. Referring to FIG. 5, one or both of the front and rear body panel webs are shifted in the longitudinal machine direction, as shown to align the maximum rises, or peaks 54, of the front and rear body panels, which also results in the alignment of the minimum rises, or valleys 56, of the front and rear body panels. For example, a first and second conveyor can simply be spaced apart so as to provide for a longer travel for one of the front and rear body panel webs.

Referring to FIG. 5, the front and rear body panel webs 92, 94 are also separated, or shifted, outwardly relative to one another in the lateral cross-direction 104 so as to form a gap 106 between the cut edges 100, 102 of the front and rear body panel webs 92, 94 at the maximum rise formed at the respective peaks 54. This separation results in the front and rear body panel webs 92, 94 being separated with no portions of either web overlapping each other. For example, in one embodiment, a first pair of rollers (not shown) can be angled or twisted to laterally spread the front and rear body panel webs 92, 94 a first amount before they are shifted in the longitudinal machine direction. A second pair of rollers (not shown) can be angled or twisted to laterally spread the front and rear body panel webs 92, 94 a second amount after they are shifted in the longitudinal machine direction. Of course, it should be understood that the front and rear body panels can be first shifted in the longitudinal machine direction the desired amount and then separated in the lateral cross direction the entire desired amount, or they can also be first separated in the lateral cross direction the entire desired amount and then shifted in the longitudinal machine direction. Various aspects of the formation of the front and rear body panel webs is further described and disclosed in U.S. patent application Ser. No. 10/261,805, filed Oct. 1, 2002, entitled “THREE PIECE DISPOSABLE UNDERGARMENT AND METHOD FOR THE MANUFACTURE THEREOF,” and assigned to Kimberly-Clark Worldwide, Inc., the assignee of the present application, the entire disclosure of which is hereby incorporated herein by reference.

After the body panel webs 92, 94 are aligned and separated, regardless of the order thereof, a plurality of crotch members 50 are positioned in the lateral cross direction so as to bridge the gaps 106 between the body panel webs 92, 94 at successive peaks 54 where the maximum rises of the body panel webs are aligned. The crotch members 50 are secured to the body panel webs 92, 94 as explained above. It should be understood that the crotch members 50 can be secured to a bodyside surface of the body panel webs 92, 94, or to the garment side thereof.

As shown in FIGS. 5 and 6, the crotch members are assembled offline and are then applied to the front and rear body panel webs 92, 94 as those webs are carried, for example, by a construction drum (not shown). In one embodiment, a web of absorbent material 108 traveling in a machine direction 90 is cut along the cross-direction 104 to form a plurality of retention elements, whether forming the entirety of the crotch member or incorporated as a component thereof. The cuts are successively angled so as to form the trapezoidal shaped retention elements, with the non-parallel edges 84 thereof lying in the cross-direction 104.

In one embodiment, shown in FIG. 6, the front and rear body panel webs 92, 94 have an identical shape. Accordingly, the retention element 70, or the crotch member 50 incorporating such a member, are successively connected to the body panel webs without rotating the retention element or crotch member. In this way, each body panel web 92, 94, once cut, serves to form alternating front and rear body panels on successive garments, with the rear body panel having the wider end of the retention member 70 secured thereto. The garments subsequently can be rotated, as desired, for packaging.

In another embodiment, shown for example in FIG. 5, where the front and rear body panel webs 92, 94 are not identical, every other retention element 70, or crotch member 50 incorporating such an element, are rotated 180° before applying the element or member to the body panel webs. The retention element or crotch member can be rotated about the machine direction axis 90, or about an axis perpendicular to the plane of the web 108, depending on the construction of the crotch member, both of which axes are non-parallel to the cross-direction axis. For example, if the crotch member such as the retention element, is symmetrical from the body side to the garment side, the crotch member can be rotated about the machine direction axis 90. If, however, the crotch member includes a number of different layers, including the retention portion, and it is important to maintain the orientation of those layers, the crotch member can be rotated 180° in the plane of the web 108 about the axis perpendicular to the web 108.

It should be understood that the retention element 70 can be cut in the trapezoidal shape and directly applied to the body panel webs as just described, or it can be cut in the trapezoidal shape and thereafter assembled into a composite crotch member, having for example and without limitation a topsheet and backsheet as described above. In addition, the retention member 70 can be rotated before being incorporated into the composite crotch member, or the crotch member can be rotated once assembled before applying it to the body panel webs 92, 94, as explained above.

In one embodiment, the crotch member is rotated using an offset cam action rotator (not shown). The rotator includes a plurality of transfer segments, which can have a vacuum applied thereto, that engage the crotch members. Coupler arms connect the transfer segments and a drive ring. The coupler arm includes a cam end having a cam follower that follows the profile of a cam mechanism. The profile of the cam mechanism can be readily changed to change the desired speed output and pitch of the crotch member. In one preferred embodiment, the rotator is configured to accelerate the crotch member. If the successive crotch members 50 are separated by a perforation, the transfer segment breaks the perforation as it engages one member and moves away from the next member, which is engaged by a next transfer segment. The rotator rotates the end portion of the transfer segment, preferably approximately 90 degrees, about a radial axis, such that the crotch member is oriented as described above as the transfer segments are rotated about a horizontal axis. The rotator, and the method for the use thereof, is further disclosed in U.S. Pat. Nos. 5,761,478, 5,759,340, and 6,139,004, and U.S. patent application Ser. No. 10/038,766, entitled “Apparatus For Applying Discrete Parts to A Moving Web,” filed Jan. 2, 2002, all of which are assigned to Kimberly-Clark Worldwide, Inc., the assignee of the present application, and the entire disclosures of all of which are hereby incorporated herein by reference. Alternatively, the crotch member can be rotated using a revolving transfer roll as shown and described in U.S. Pat. No. 4,608,115, which is assigned to Kimberly-Clark Worldwide, Inc., the assignee of the present application, and which is hereby incorporated herein by reference in its entirety.

After the crotch members 50 are secured to the body panel webs 92, 94 across the gap 106, the absorbent garments, and in particular the crotch members, are successively folded, for example with a helical folder, such that the front and rear body panel webs 92, 94 are positioned in an overlapping, or overlying relationship, preferably with the outer edges 96, 98 aligned. In various alternative embodiments, the body panel webs 92, 94 can be secured, for example by bonding, along a lateral cross direction at the area of minimum rise, or valleys 56 to form side seams. The front and rear body panel webs 92, 94 are then cut along the lateral cross direction along the side seam to form a plurality of discrete absorbent garments, each having a front and rear body panel 4, 6. Alternatively, the front and rear body panel webs 92, 94 can be first cut at the area of minimum rise, or valleys 56, and the crotch member 50 thereafter folded. Various refastenable fastening members can be applied to the front and rear body panels 4, 6 or front and rear body panel webs 92, 94 before or after the various cutting and folding operations. In yet another preferred embodiment, as explained above, the absorbent garment can be configured with side seams which secure the front and rear body panels, and refastenable fastening members, which bridge lines of weakness formed in one or the other of the body panels.

The elastic elements 36 are preferably incorporated into the web of body panel material prior to cutting the web to form the front and rear body panel webs 92, 94. If leg elastics are desired, preferably they are integrated into the web along a pattern that will follow the cut edge 100, 102 on each of the front and rear body panel webs 92, 94. Of course, it should be understood that the elastics could be secured to the body panel webs after they are formed by the die cutter, or other cutting operation. In addition, the elastics can be non-continuous and intermittently spaced along the longitudinal direction such that they are incorporated along only the leg opening portions 18, 26 of the body panel webs and body panels.

Although the present invention has been described with reference to preferred embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

What is claimed is:

1. An absorbent garment comprising:
a front body panel comprising a terminal waist edge and a terminal crotch edge;
a rear body panel comprising a terminal waist edge and a terminal crotch edge; and
a crotch member extending between and coupled to said front and rear body panels, said crotch member comprising a retention element having a trapezoidal shape.

2. The absorbent garment of claim 1 wherein said crotch edge of said rear body panel is spaced apart from said crotch edge of said front body panel so as to form a gap therebetween, and wherein said crotch member spans said gap

3. The absorbent garment of claim 1 wherein said retention element consists essentially of an absorbent material.

4. The absorbent garment of claim 1 wherein said retention element comprises a superabsorbent material.

5. The absorbent garment of claim 1 wherein said retention element has first and second parallel terminal edges and non-parallel, linear side edge, wherein said first terminal edge is shorter than said second terminal edge.

6. The absorbent garment of claim 5 wherein said first terminal edge overlies said front body panel and wherein said second terminal edge overlies said rear body panel.

7. The absorbent garment of claim 1 wherein said terminal crotch edges of said front and rear body panels are curvilinear.

8. The absorbent garment of claim 1 wherein said crotch member further comprises a top sheet and a back sheet, wherein said retention element is sandwiched between said top sheet and said back sheet.

9. A method of manufacturing an absorbent garment comprising:
moving first and second webs of body panel material in a first machine direction;
moving a web of absorbent material in a second machine direction;
cutting said web of absorbent material and thereby forming a plurality of trapezoidally shaped retention elements, each of said plurality of retention elements having non-parallel side edges; and
successively coupling said plurality of retention elements to said first and second webs with said side edges extending in a cross-direction.

10. The method of claim 9 wherein said first and second machine directions are parallel.

11. The method of claim 9 wherein each of said plurality of retention elements comprises a first terminal edge and a second terminal edge, wherein said first terminal edge is shorter than said second terminal edge, and wherein said first and second terminal edges of each of said plurality of retention elements are positioned adjacent said second and first terminal edges respectively of a next adjacent retention element.

12. The method of claim 9 wherein said cutting said web of absorbent material comprises cutting said web of said absorbent web in a cross direction and thereby forming said non-parallel side edges.

13. The method of claim 9 further comprising rotating every other one of said plurality of said retention elements prior to said successively coupling said plurality of retention elements to said first and second webs.

14. The method of claim 13 wherein said rotating every other one of said plurality of said retention elements comprises rotating every other one of said plurality of said retention elements about an axis other than a cross direction axis.

15. The method of claim 9 further comprising sandwiching each of said plurality of retention elements between a backsheet and a topsheet, wherein said successively coupling said plurality of retention elements to said first and second webs comprises successively connecting one of said back sheet and said top sheet to said first and second webs.

16. The method of claim 9 wherein said first and second webs have first and second crotch edges respectively, wherein said first crotch edge is spaced from said second crotch edge.

17. The method of claim 16 wherein said first and second crotch edges each comprise alternating peaks and valleys, wherein said peaks of said first crotch edge are aligned in the cross direction with the peaks of said second crotch edge.

18. The method of claim 9 wherein said retention element consists essentially of an absorbent material.

19. The method of claim 9 wherein said retention element comprises a superabsorbent material.

20. A method of manufacturing an absorbent garment comprising:
moving first and second webs of body panel material in a first machine direction, wherein said first and second webs have first and second crotch edges respectively, wherein said first crotch edge is spaced from said second crotch edge so as to form a gap therebetween;
moving a web of absorbent material in a second machine direction;
cutting said web of absorbent material and thereby forming a plurality of trapezoidally shaped retention portions, each of said plurality of retention portions having non-parallel side edges, a first terminal edge and a second terminal edge, wherein said first terminal edge is shorter than said second terminal edge, and wherein said first and second terminal edges of each of said plurality of retention elements are positioned adjacent said second and first terminal edges respectively of a next adjacent retention element;
successively rotating every other one of said plurality of said retention elements approximately 180 degrees about an axis other than a cross direction axis; and
successively coupling said plurality of retention portions to said first and second webs with said side edges extending in a cross-direction, wherein said retention portion bridges at least a portion of said gap between said first and second webs.

21. The method of claim 20 further comprising sandwiching each of said plurality of retention elements between a backsheet and a topsheet, wherein said successively coupling said plurality of retention elements to said first and second webs comprises successively connecting one of said back sheet and said top sheet to said first and second webs, wherein at least one of said back sheet and said top sheet bridged said gap between said first and second webs.

22. The method of claim 20 wherein said first and second crotch edges each comprise alternating peaks and valleys, wherein said peaks of said first crotch edge are aligned in the cross direction with the peaks of said second crotch edge.
